(19) United States
(12) Patent Application Publication
(10) Pub. No.: US 2025/0387156 A1
(43) Pub. Date: Dec. 25, 2025

(54) INTERVENTIONAL GUIDANCE

(71) Applicant: MEDTRONIC, INC., MOUNDS VIEW, MN (US)

(72) Inventors: Daniel C. SIGG, SAINT PAUL, MN (US); Lars M. MATTISON, BLAINE, MN (US)

(21) Appl. No.: 18/879,530

(22) PCT Filed: Jul. 5, 2023
(86) PCT No.: PCT/US2023/069640

§ 371 Date: Dec. 27, 2024

Related U.S. Application Data
(60) Provisional application No. 63/358,380, filed on Jul. 5, 2022.

(51) Int. Cl. A61B 18/14; A61B 18/00; A61B 18/02; A61B 34/10

(52) U.S. Cl. CPC A61B 18/1492; A61B 18/02; A61B 34/10; A61B 2018/0022; A61B 2018/00351; A61B 2018/00577; A61B 2018/00613; A61B 2018/00642; A61B 2018/00791; A61B 2018/00839; A61B 2018/00875; A61B 2018/0212; A61B 2018/1407; A61B 2018/1467; A61B 2034/104; A61B 2034/105; A61B 2034/107

(57) ABSTRACT

An example computer-implemented method includes determining a location of a probe relative to patient anatomy, in which the probe includes an emitter adapted to deliver energy. The method also includes computing a virtual spatial projection of an energy field for the emitter based on the location of the probe and at least one operating parameter for the emitter. The method also includes generating guidance for performing an intervention with the probe based on the virtual spatial projection.

### FIELD

The present technology is generally related to generating guidance for applying an intervention to an anatomical target.

### BACKGROUND

Various procedures have been developed to analyze, diagnose and/or treat various organ systems including electrophysiological conditions of the cardiovascular system, central nervous system or peripheral nervous system. For example, energy can be applied from a catheter or other probe device to ablate target tissue, such as through a respective ablation modality. The efficacy of a given treatment can be reduced if the given treatment adversely affects neighboring tissue or other anatomical features that might be sensitive to the treatment.

### SUMMARY

The techniques of this disclosure generally relate to generating guidance for a surgical intervention.

In one aspect, the present disclosure provides a computer-implemented method includes determining a location of a probe relative to patient anatomy, in which the probe includes an emitter adapted to deliver energy. The method also includes computing a virtual spatial projection of an energy field for the emitter based on the location of the probe and at least one operating parameter for the emitter. The method also includes generating guidance for performing an intervention with the probe based on the virtual spatial projection. In another aspect, one or more non-transitory machine-readable media have instructions, which, when executed by a processor, perform the method.

In yet another aspect, the disclosure provides a system that includes an elongated probe comprising an emitter adjacent a distal end thereof. The emitter can be configured to deliver energy based on at least one operating parameter thereof. The system also includes non-transitory memory configured to store data and machine-readable instructions, and one or more processors are adapted to access the memory and execute the instructions. The processor thus can determine a location of the probe relative to patient anatomy based on location data and geometry data. The location data represents spatial coordinates of the probe, and the geometry data spatially represents at least a target region of the patient anatomy. The processor can also determine a virtual spatial projection of an energy field for the emitter relative to the patient anatomy based on the location of the probe, emitter data and at least one operating parameter for the emitter. The emitter data can describe energy field properties for the emitter. The processor can also generate guidance based on the virtual spatial projection.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram of an example system that can be implemented for providing interventional guidance.

FIG. 2A, 2B and 2C are schematic diagrams showing a graphical visualization of interventional guidance for different operating parameters.

FIGS. 3A and 3B are schematic illustrations showing an example of using of an ablation catheter.

FIG. 4 is a schematic illustration showing an example of a graphical representation of output guidance superimposed on a graphical representation of anatomy.

FIG. 5 is a schematic illustration showing another example of a graphical representation of output guidance superimposed on a graphical representation of anatomy.

FIGS. 6A, 6B, and 6C are graphs showing examples of energy fields that can be generated for different configurations of ablation electrodes.

FIGS. 7A, 7B and 7C are graphs showing examples of energy fields that can be generated for other configurations of ablation electrodes.

FIG. 8 is a flow diagram of an example method that can be implemented for evaluating impact of one or more interventions.

### DETAILED DESCRIPTION

This disclosure relates to generating guidance for applying an intervention to an anatomical target. For example, the intervention can include delivery of energy, such as to alter the conductivity of a target region of tissue within the patient's body. The type of energy being delivered can vary depending on the target region and desired therapeutic or sub-therapeutic effect. Examples of energy types that can be applied include electrical energy, thermal energy, ultrasound energy, electromagnetic radiation (e.g., optical energy, such as laser ablation) and electrical energy (e.g. irreversible electroporation). In examples described herein, the energy is delivered by an emitter device configured to ablate target tissue residing in a region of interest, such as by performing radiofrequency ablation, cryoablation, laser ablation or pulsed field ablation (PFA). For example, PFA is a non-thermal an ablation modality involving the use of pulsed electric fields to achieve cell death via mechanisms of irreversible electroporation.

The systems and methods described herein can provide guidance to ensure that sufficient energy is delivered accurately to the target tissue to achieve the desired therapeutic effect (e.g., ablation) or sub-therapeutic effect. Additionally, systems and methods described herein can provide guidance to enable the delivery of energy to be adjusted (e.g., automatically or responsive to a user input) to reduce or prevent delivery of energy to one or more non-target anatomical tissue regions. For example, the location and level of energy being delivered can be shaped to avoid non-target regions including one or more anatomical features (e.g., phrenic nerves, esophagus and/or other structures) particularly susceptible or sensitive to damage during such treatment. Additionally, or as an alternative, the energy can be applied to tissue as part of a pre-treatment process (e.g., by heating or cooling tissue) to change conductivity of such tissue to facilitate a subsequent treatment of the tissue or neighboring tissue.

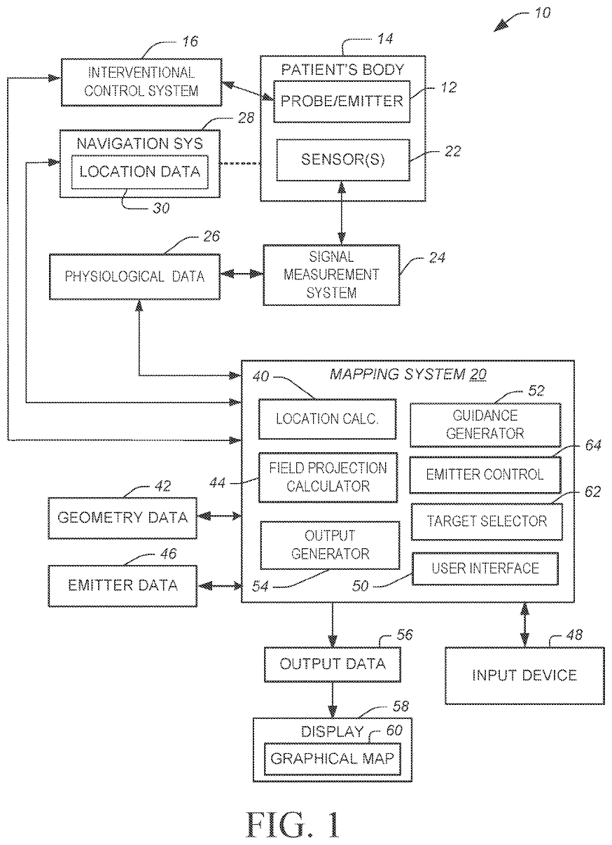

FIG. 1 illustrates a system 10 configured to provide interventional guidance. The system 10 includes an emitter 12 that can be located adjacent a distal end of a probe, such as a catheter, guidewire or a cannula. The emitter 12 is configured to deliver energy based on at least one operating parameter, which can vary depending on the type of energy being delivered. The type of emitter also can depend on the type of energy to be delivered. In an example, a cardiac catheter carrying the emitter 12 can be inserted into a femoral vein (or other known entry point) and advanced to position the emitter 12 at a desired position within the patient's body 14, such as a target region of the heart. In other examples, the probe and emitter 12 can be adapted to be inserted in the body 14 and advanced to one or more locations for delivering energy to one or more other anatomical targets, such as part of the central nervous system, peripheral nervous system, the digestive system or the like. Thus, while many examples described herein relate to providing guidance for performing a cardiac intervention, the systems and methods described herein are applicable to other anatomical features. The system 10 can use any number of one or more probes and emitters 12 concurrently, which can be moved independently to different sites within the patient's body 14.

The system 10 can include an interventional control system 16 configured to control the operation of the emitter 12. For example, the control system 16 includes hardware and/or software configured to set one or more operating parameters and/or sourcing energy for controlling energy to be delivered by the emitter 12. The emitter is thus configured to deliver corresponding energy (e.g., electrical energy, thermal energy, ultrasonic energy, electromagnetic energy, etc.) based on the interventional control. The catheter or probe can be moved manually, robotically assisted or fully robotically to control where the emitter 12 is positioned.

For the example of electrical energy, the parameters can include energy level (e.g., current and voltage), pulse width, duty cycle, device shape, and repetition rate. For the example of a laser intervention providing electromagnetic radiation (e.g., coherent or laser light) the parameters can include energy level, duration, wavelength and repetition rate. For the example of a radiofrequency (RF) ablation intervention the parameters can include a number of electrodes to be activated, device shape, energy level (e.g., current and voltage), duration, and repetition rate. For the example of a pulsed field ablation (PFA) intervention, the parameters can specify a number of electrodes, energy level (e.g., current and voltage), waveform composition, device shape, pulse as well as pulse train number and duration. The parameters can control whether the PFA intervention is permanent or reversible. Other parameters can be used and configured according to the type and configuration of emitter 12, and desired outcome. The control system can fix parameters during delivery of a respective intervention or the control system 16 can vary one or more parameters during the application of the intervention. The device shape also provides a variable parameters that can vary during an intervention, as the shape can deform and/or deflect during use, such as in response to contacting tissue.

The control system 16 can set the parameters and apply an intervention based on automatic, manual (e.g., user input) or a combination of automatic and manual (e.g., semiautomatic controls). One or more sensors (e.g., on the emitter or catheter-not shown) can also communicate sensor information (e.g., feedback) back to the control system 16. The sensor information can describe a sensed condition of the emitter 12 and/or the tissue to which the intervention is being applied. The control system 16 can also be coupled to a mapping system 20, such as to receive instructions, such as commands (e.g., to set operating parameters) or to trigger the control system 16 to apply the intervention. The control system 16 can also provide interventional data to the mapping system 20, such as describing parameters used for application of the intervention and a timestamp describing when the respective intervention is applied.

The system 10 can also include one or more sensors 22. For example, the sensor 22 can include one or more electrodes adapted to measure electrophysiological signals from the body 14, such as cardiac electrophysiological signals of the heart. The sensors 22 can be carried by the probe (e.g., catheter), and configured (e.g., as contact or non-contact electrodes) to measure cardiac electrophysiological signals of the heart. Such electrodes can be used to perform mapping for an epicardial or endocardial cardiac surface. Additionally, or alternatively, the sensors 22 can include a distributed arrangement of multiple body surface electrodes (e.g., about 50, 100, 250 or more sensors) configured to be positioned on an outer surface of the patient's body 14. In an example, an arrangement of the sensors 22, constituting body surface electrodes, are distributed completely around the thorax, such as can be mounted to a wearable garment (e.g., vest) in which each of the electrodes has a known location in a given coordinate system. For example, body surface electrodes can be implemented as a non-invasive type of sensor apparatus as disclosed in U.S. Patent Publication No. 2013/0281814, entitled Multi-Layered Sensor Apparatus. Other configurations and numbers of body surface electrodes 22 could be utilized in other examples. A signal measurement system 24 can be coupled to the sensors 22 and configured to receive electrophysiological signals from the sensors. The signal measurement system can also include hardware and/or software configured to perform signal processing (e.g., amplification, filtering etc.) and provide corresponding physiological data 26 representative of the measured electrophysiological signals over time.

In some examples, in addition to measuring electrophysiological signals, the one or more sensors 22 are configured to measure one or more other conditions, including physiological conditions (e.g., respiration), environmental conditions (e.g., temperature and/or pressure) and/or contact between the probe/emitter 12 and tissue (e.g., based on measured force or impedance). In an example, the other sensors 22 can include a temperature sensors configured to measure tissue temperature (e.g., esophageal temperature). The sensors 22 can be integrated with the probe/emitter 12, such a part of an electrode structure of an ablation catheter, or one or more such sensors can be separate from the probe/emitter. The other condition measurements can be stored as part of the data 26 with time stamps or other information to enable further processing and analysis with the electrophysiological data.

As a further example, the system 10 include a navigation system 28 configured to localize the spatial position of the emitter 12 (or a catheter to which the emitter is coupled). For example, the navigation system provides location data 30 representative of a location for the emitter 12. In some examples, the location data 30 provides spatial coordinates for one or more sensors (e.g., electromagnetic coil sensors, shown as sensors 22) having a known fixed location relative to the emitter 12, which is used to derive spatial coordinates for the emitter 12. The location data 30 can be stored in memory of the navigation system 28 and/or memory of the mapping system 20. The location data 30 can represent a three-dimensional spatial position (e.g., spatial coordinates) and orientation of the emitter 12. Alternatively, the location data 30 can represent the location of a location sensor or other known location on the probe carrying the emitter, and the spatial location emitter and/or other sensors can be derived readily from the location data 30. In examples where the emitter 12 and one or more of the sensors 22 are integrated in a single device (e.g., an ablation catheter), the same location data 30 can represent the spatial position of both. In examples where the sensors 22 and the emitter 12 are implemented in independently movable structures, separate location data 30 can be generated to represent respective spatial positions.

The location data 30 can be provided in a coordinate system of the patient's body 14 or a coordinate system of the navigation system 28. For example, the spatial location of the emitter 12, which is described by or derived from the location data 30, can be registered with respect to anatomical geometry of the patient's body 14. The registration can be repeated in response to detecting changes in the location data, such as the probe carrying the emitter is moved within the patient's body. In some examples, the navigation system 28 can also generate the location data 30 to include the location of one or more non-invasive sensors 22, such as can be distributed across an outer surface of the patient's body (e.g., on the thorax). For example, the emitter 12 as well as sensors 22 can be sensorized (e.g., include navigation sensors mounted located at known locations) to enable the navigation system 28 to track respective spatial positions and orientation in real time.

Useful examples of the navigation system 28 include the STEALTH STATION navigation system (commercially available from Medtronic), the CARTO XP EP navigation system (commercially available from Biosense-Webster) and the ENSITE NAVX visualization and navigation technology (commercially available from St. Jude Medical); although other navigations systems could be used to provide the navigation data representative of the spatial position for emitter 12 and associated probe. Another example of a navigation system that can be utilized to localize the position of the invasive electrodes is disclosed in U.S. Pat. No. 10,323,922, issued Jun. 18, 2019 Aug. 29, 2014, and entitled LOCALIZATION AND TRACKING OF AN OBJECT. For example, a probe (e.g., catheter) can include an emitter 12 having one or more emitter elements (e.g., electrode(s), cryoballoon, optical fiber laser, etc.) disposed at known locations of the probe. The probe can be used to position each such emitter with respect to the heart or other anatomical structure, and the navigation system 28 can provide corresponding location data 30 representing three-dimensional coordinates for the emitter.

The system 10 includes a computing apparatus having one or more processors configured to access memory that stores data and instructions. The processor(s) can access and execute instructions corresponding to the functions and methods implemented by the mapping system 20. The mapping system 20 thus includes instructions executable by the one or more processors of the computing apparatus to perform the functions described herein.

In the example of FIG. 1, the mapping system 20 includes a location calculator 40 programmed to determine a location of the probe relative to patient anatomy based on the location data 30 and geometry data 42. As described above, the location data 30 represents three-dimensional spatial coordinates of the probe carrying the emitter or the emitter itself. The spatial coordinates can be provided in the coordinate system of the navigation system or have been registered with respect to the patient's body 14. The geometry data 42 can be generated (e.g., by geometry determining code-not shown) to spatially represent at least a region of the patient anatomy where the emitter is to apply energy.

As an example, the geometry data 42 can be anatomical geometry derived from imaging data acquired by a medical imaging modality, such as single or multi-plane x-ray, computed tomography (CT), magnetic resonance imaging (MRI), ultrasound, positron emission tomography (PET), single-photon emission computed tomography (SPECT) and the like. The locations of sensors 22 and one or more surfaces of interest can be identified in a respective coordinate system of the acquired images (e.g., in the coordinate space of the body 14) through appropriate image processing, including extraction and segmentation. For instance, segmented image data can be converted into a two-dimensional or three-dimensional graphical representation that includes the volume of interest for the patient. Appropriate anatomical or other landmarks can be identified in the geometry data 42 to facilitate spatial registration of the location data 30 and sensors. The identification of such landmarks can be done manually (e.g., by a person via image editing software) or automatically (e.g., via image processing techniques). In one example, an anatomical model can be constructed based on imaging data obtained (e.g., by a medical imaging modality) for the patient to provide spatial coordinates or a model to describe the surface of interest as well as any sensors 22 on or in the body at the time of imaging. In some examples, the geometry data can also include an impedance and/or conductivity map for the patient's anatomy, such as can be determined based on image analysis and/or electrical measurements across the patient's body.

The location calculator 40 can include a registration method programmed to spatially register the location data with the geometry data. For example, the registration method is programmed to transfer the location data 30 and the geometry data 42 into a common coordinate space (e.g., spatial domain), which can be the coordinate space of the location data 30, the geometry data 42 or another common three-dimensional coordinate space. As a result, the location calculator 40 can provide the location (spatial coordinates) of the probe/emitter 12 in relative to patient anatomy described by the geometry data.

The mapping system 20 also includes a field projection calculator 44 programmed determine a virtual spatial projection of an energy field for the emitter 12 relative to the patient anatomy based on the location of the probe (e.g., determined by the location calculator 40), emitter data 46 and at least one operating parameter for the emitter. In some examples, the virtual spatial projection can also vary depending on the properties of the tissue, such as tissue impedance or conductivity, thickness and the like. The emitter data 46 describes field properties for the emitter 12, which can vary depending on the operating parameter(s) and configuration of the emitter 12. One or more of the operating parameters and configuration of the emitter can be selected in response to a user input, such as using a user input device 48 (e.g., mouse, keyboard, touchscreen interface, gesture interface or the like) to interact and provide user instructions to a user interface 50. The user interface 50 can be programmed to access control functions of the methods implemented by the mapping system 20, such as through a set of defined application programming interfaces (APIs). The field projection calculator 40 thus determines the virtual spatial projection as a three-dimensional volume representing the energy field, which can vary based on emitter operating parameters, relative emitter location (e.g., distance from tissue), shape/configuration of the emitter and properties of surrounding tissue (e.g., conductivity and/or impedance). As any one or more such parameters change during the intervention, such as in response to a user input or contact of the emitter with tissue, the resulting virtual spatial projection likewise can change.

As an example, the emitter data 46 can store a field model describing a three-dimensional energy field that is delivered by the emitter 12 for a set or one or more operating parameters. The field model can include a mathematical model that is used by the field projection calculator to determine the virtual spatial projection of an energy field for the emitter 12. In one example, a virtual spatial projection can be represented in three-dimensional space as a volume including a boundary of reversible electroporation threshold (calculated in field strength, V/cm) and/or irreversible electroporation threshold (calculated in field strength, V/cm). The parameter of the field strength would receive inputs from a PFA system (or entered via a user interface), including number of electrodes energized, voltage, current, as well as pulse wave parameters. In another example, the virtual spatial projection can be represented in three-dimensional space as a volume including a boundary, and can be registered to the spatial domain of the emitter, such as determined by the location calculator 40. The three-dimensional configuration can vary depending on the operating parameter(s). In some examples, where the virtual spatial projection overlaps tissue in a way that would affect the shape or level of energy in the field represented by the virtual spatial projection, the field projection calculator 44 can be programmed to adjust the virtual spatial projection accordingly. As described above, for a given multi-electrode configuration, the number of active electrodes and operating parameters for each active electrode can be specified in response to a user input, and used by the field projection calculator 44 in computing the virtual field projection. As the shape or configuration of the emitter changes, such as deflection/deformation responsive to contacting tissue, such deflection/deformation can be detected (e.g., as a parameter change), which is reflected in the virtual field projection that is computed.

In some examples, the emitter data 46 includes a library of emitter models for a plurality of different emitters, which can include different configurations of a common type of emitter or different types of emitters (e.g., RF ablation electrodes, cryoballoons, PFA electrodes). For the example where the emitter 12 is implemented as RF and/or PFA electrodes, the available electrode models can include configurations ranging from a single electrode (corresponding to a single point) or an arrangement of electrodes (such as disposed along a straight or curved shaft) or three-dimensional electrode configurations (e.g., representing a volumetric arrangement of electrodes, such as on a basket, sphere or other 3D shape). Respective emitter models can be generated for one or more manufactures' product lines to facilitate selecting the correct configuration matching the emitter 12 that is being used. For example, a user can select a given manufacturer and model number of emitter from drop-down user interface, and the field projection calculator 44 can access corresponding emitter data for the selected emitter for use when computing the virtual field projection of the emitter 12.

The mapping system 20 also includes a guidance generator 52 configured to generate guidance based on the virtual spatial projection. For example, the guidance generator is programmed to spatially register the virtual spatial projection with a patient anatomy based on the determined location of the emitter, and provide the spatially registered features as guidance data to an output generator. The output generator 54 can be programmed to provide output data 56 that include graphics text and other information that is rendered graphically on a display 58. The display 58 can include a screen, wearable augmented reality glasses, a heads-up display or the like. The display 58 is configured to display a graphical visualization based on the output data 56, such as including a rendering of a graphical map 60 presenting a graphical representation of the virtual field projection superimposed relative to a graphical representation of the patient's anatomy. In some examples, the graphical map 60 includes a graphical representation of the virtual field projection superimposed relative to a graphical representation an electroanatomic map, such as a potential map of a cardiac surface. The electroanatomic map can be generated based on electrophysiological signals measured invasively, non-invasively from the body surface or a combination of invasive and non-invasive electrophysiological signal measurements. In an example, the mapping system 20 includes code programmed to reconstruct electrophysiological signals onto a cardiac surface based on the electrophysiological data and the geometry data, such as by solving the inverse problem. For example, the mapping system 20 can be programmed to compute the reconstructed electrophysiological signals according to any of the approaches as described in U.S. Pat. Nos. 6,772,004 or 7,983,743.

The portions of anatomy within the visualization provided by the graphical map 60 can be updated based on the location data 30, such as to maintain the emitter within the visualization. It is to be understood that, at this stage of the workflow, the operating parameter(s) used by the field projection calculator 44 are used for purposes of computing the virtual spatial projection of the energy field and generating corresponding guidance in the absence of controlling the emitter to deliver actual energy. Thus, the guidance affords the user an advanced preview of how the energy (if applied based on the current operating parameter(s)) might affect the tissue. The output generator 54 can further generate the output data 56 to include information (e.g., text and/or graphics) based on other data representing one or more other conditions measured by the sensors 22, such as described herein (e.g., temperature, pressure, contact, etc.). The other sensed conditions thus can be presented on the display 60 with graphical representation of the virtual field projection (e.g., in the same or different display window) to provide additional guidance into the process, including before, during and after delivering energy. In an example, the guidance generator 52 and/or output generator 54 are programmed to cooperate to project information captured or derived from one or more such other sensors 22 (e.g., data 26) onto the virtual spatial projection. In another example, the output generator 54 and/or location calculator 40 can be programmed to determine the location of electrodes or other sensors 22 based on the data 26 and 30 to display a representation of catheter geometry projected onto a graphical representation of patient anatomy, such as showing which electrodes are in contact with tissue.

In some examples, the mapping system 20 includes a target selector 62. The target selector 62 can be programmed to define one or more target and/or non-target regions within the patient's body. For example, one or more anatomical features to be avoided can be determined, such as by default set of rules or specified manually in response to a user selection in an image or graphical map through the input device 48. A target or non-target region can be an anatomical landmark or other identified region of tissue (e.g., phrenic nerves, pulmonary veins and/or other structures) considered susceptible or sensitive to damage during such treatment. The guidance generator 52 can further be programmed to provide output guidance as a graphical visualization that includes the virtual spatial projection, a graphical representation of each non-target region and a graphical representation of one or more target region within the patient's body. As an example, the guidance generator 52 can be programmed to generate an output to indicate whether the at least one non-target region resides within the three-dimensional volume of the virtual spatial projection. The output generator 54 can provide this as a notification in the output data 56, such as can be presented on the display as a graphical representation, text representation and/or audible sound. The designated non-target region can be color coded or otherwise graphically differentiated from other anatomical structures.

In another example, a target area can be identified via one or more physiological stimulation mechanisms. For example, a pacing stimulus can be applied to an indwelling electrode to stimulate a nerve of interest, such as the phrenic nerve. If a physiological pacing response is generated with a certain threshold (in volts) of stimulation, the distance to the target to be avoided can be estimated, and projected as an anatomical structure onto the electro-anatomical map.

As a further example, when the emitter includes one or more RF ablation or PFA electrodes, the energy field can be representative of an electrical field. In such example, the shape of the electric field and relative penetration depth of current densities can be programmed into the emitter model over a set of operating parameters. For example, if the shape of the electric field and relative penetration depth of current densities into tissue are strong enough, the field could damage non-target tissue (e.g., nerve tissue, such as the phrenic nerve). Therefore, the guidance generator 52 can be programmed to suggest or specify one or more operating parameters to avoid potential damage to non-target regions, such as by identifying one or more operating parameters (e.g., power, duration of energy delivery, which electrodes should be active and the like). In some examples, the guidance generator 52 can provide a warning and/or specify a distance between a field projection or probe and any defined non-target that should be avoided. The warning can be generated if the field projection or probe is within a threshold distance from the non-target. The threshold distance can be a default value or be user programmable in response to user input. Additionally, the user can manually adjust (e.g., tune in response to a user input) one or more parameters of the emitter in response to determining that the at least one non-target region resides within the three-dimensional volume of the virtual spatial projection. In another example, the guidance generator 52 can be enabled (e.g., in response to a user input) to automatically adjust (e.g., tune) one or more operating parameters of the emitter so the target region resides within the three-dimensional volume of the virtual spatial projection and the at least one non-target region resides outside of the three-dimensional volume of the virtual spatial projection.

In examples where the emitter is configured to perform reversible PFA, the guidance generator 52 can be programmed to generate the field projection to represent the region of tissue that is to exhibit reversible electroporation, the stimulation region of tissue or both the region of tissue exhibiting reversible electroporation and the stimulation region. The stimulation region is usually larger than the reversible region. For example, the guidance generator 52 can be programmed to assign weights (e.g., in response to a user input) to specify an importance of stimulation and reversible regions, which can be used to adjust and/or augment the delivery parameters for the energy field.

In another example, a user can also move the probe and emitter to a different location, and the location calculator 40, field projection calculator 44 and guidance generator 52 will update respective computations resulting in updated guidance being generated and provided to the user. For example, the field projection calculator 40 will update the three-dimensional volume for the virtual spatial projection based on the location of the emitter (e.g., relative location determined by the location calculator) and/or the adjusted operating parameter(s) for the emitter.

After the user is satisfied with the guidance provided showing the virtual field projection on a graphical representation of the patient's anatomy, the user can set (e.g., lock in) one or more operating parameters through an emitter control function 64. Alternatively, the current (e.g., most recent) operating parameters can be automatically selected for the current procedure until or unless modified by the user in response to a user input. The emitter control function 64 is configured to provide instructions to the control system 16 (e.g., through a wired or wireless communication link) specifying operating parameters for controlling the emitter 12 to deliver energy to the target region within the patient's body. For example, the guidance generator 52 can further be programmed to supply the control function with the set of operating parameters used to provide the virtual field projection, such as automatically or in response to a user input to trigger energy delivery.

As mentioned, one or more sensors 22 can measure electrophysiological signals that are stored as the physiological data 26. In an example, the physiological data 26 includes electrophysiological measurements over a first time interval before delivery of the energy and over a second time interval after the delivery of the energy. The sensors can also measure other conditions, such as described herein. The output generator 54 can be programmed (in response to a user input or automatically) to provide the output data 56 to include graphical representations of the physiological data 26 for the first and second time intervals. In one example, the graphical representations of the physiological data 26 for the first and second time intervals can be provided as comparative electrogram waveforms. In another example, the graphical representations of the physiological data 26 for the first and second time intervals can be provided as comparative electroanatomical maps of electrophysiological signals that have been reconstructed onto a cardiac surface. As a result, the graphical representations can provide a side-by-side comparison of the physiological signal measurements (e.g., electrophysiological measurements and/or other physiological data) before delivery and after delivery of the energy by which a user can confirm whether a desired therapeutic effect has been achieved.

In a further example, the field projection calculator can be programmed to compute a plurality of virtual spatial projections for different values of operating parameters for the emitter. The guidance generator 52 and output generator 54 can generate respective output guidance, such as showing a graphical representation of the virtual field projection superimposed on a graphical representation of patient anatomy for each of different operating parameter values. Respective graphical outputs can be rendered in separate windows of the display 58 concurrently for comparison. A given graphical representation of the virtual field projection can be selected from among those that have been generated, such as automatically or in response to a user input selection. In response, to the selected graphical representation of the virtual field projection, respective operating parameters of the emitter 12 can be set to a corresponding value based on the selected given virtual spatial projection. In response, the emitter control 64 can instruct the control system 16 to deliver energy from the emitter based on the setting. A user can also be afforded an opportunity to confirm (or reject) instructions for the emitter to deliver the requested energy, such in response to a user input through the input device 48 or through a button or other control device on the control system 16. As a result of using the systems and methods, improved treatment strategies can be determined and a desired therapeutic effect (e.g., tissue ablation) can be achieved more efficiently and with reduced injury.

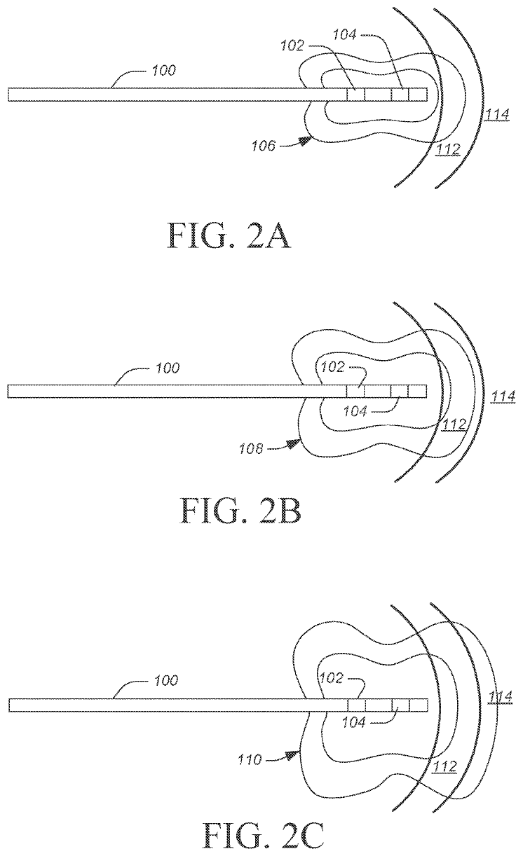

FIGS. 2A, 2B and 2C depict a representation of a probe (e.g., a catheter) 100 having an emitter located adjacent a distal end of the probe. In these examples, the emitter includes a plurality of electrodes 102 and 104 showing different examples of virtual field projections 106, 108 and 110 for an energy field (e.g., an electric field) based on respective emitter operating parameters. Any number and distribution of electrodes can be used.

For example, FIGS. 2A, 2B and 2C demonstrate fields of increasing size (e.g., 3D volume) due to increase electrical power (e.g., voltage and/or current) applied to the respective electrodes 102 and 104. As described herein, the same or different power level can be used for each of the electrodes, such as in response to a user input, based on which the field projection calculator 44 determines the respective virtual field projections 106, 108 and 110. In the case of pulsed field ablation, such field distribution could also be changed if the number of pulsed fields, or one or more of the pulse wave parameters of the pulses is being modulated.

FIGS. 2A, 2B and 2C also show target and non-target tissue regions 112 and 114, respectively, located near the distal end of the probe 100. Additionally, each of the fields includes two discrete power levels, such as can be represented based on volts per centimeter (V/cm) determined for each of the respective virtual field projections 106, 108 and 110. In the example of FIGS. 2A and 2B, at least a portion of the target region 112 resides within the respective virtual field projections 106 and 108, but the non-target region 114 reside outside of the volume of the virtual field projections 106 and 108. In contrast, in FIGS. 2C, both at least a portion of the target region 112 and non-target region 114 reside within the virtual field projection 110. Thus, the guidance generator 52 can provide output guidance (e.g., one or more graphical visualization, such as graphical maps) to indicate that using operating parameters associated with the virtual field projection 110 might have less desirable outcome than using operating parameters associated with virtual field projection 106 and 108. While the examples of FIGS. 2A, 2B and 2C show a single catheter being used, in other examples, two or more catheters can be used concurrently for energy delivery to generate a composite field projection representative of the combined fields, which can be updated in real time between the energy delivery catheters.

In some examples, the probe 100 can further include one or more sensors (e.g., electrodes or other types of sensors) to detect contact with tissue. In one example, the sensors can measure contact impedance, and the measured contact impedance can be included on a graphical output in combination with (e.g., superimposed onto) the virtual field projections, such as to visualize which electrodes are in contact with tissue. In another example, the sensors can include one or more temperature sensors, and sensed temperature can be included on a graphical output in combination with (e.g., superimposed onto) the virtual field projections, such as to visualize a temperature rise from a sub-therapeutic RF energy delivery. By displaying the sensor information (e.g., contact impedance and/or temperature) in conjunction with the field projections, as described herein, the user is afforded additional insight into the procedure and expected outcomes, which can reduce time and improve treatment accuracy.

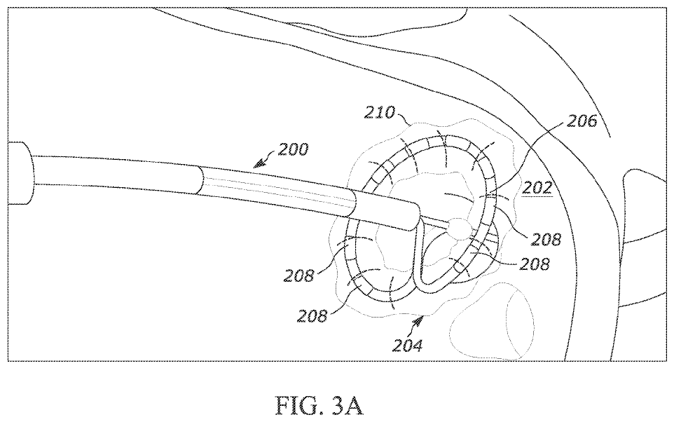

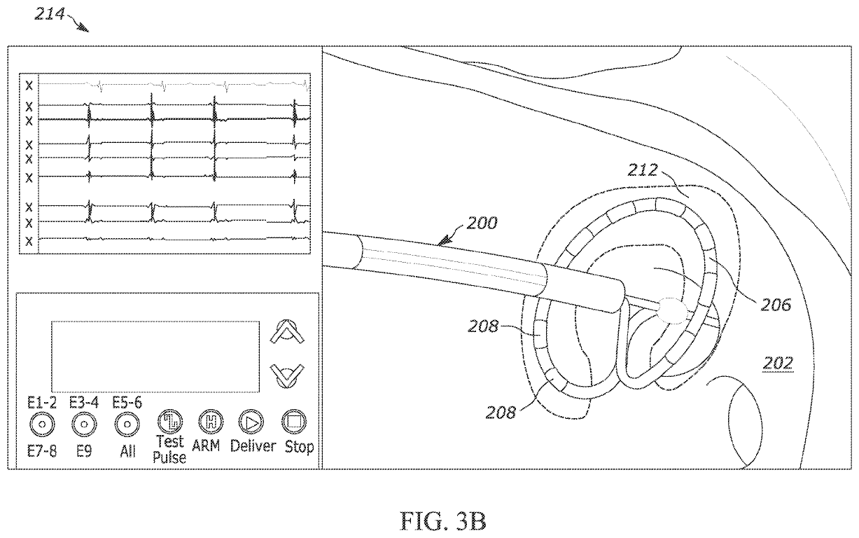

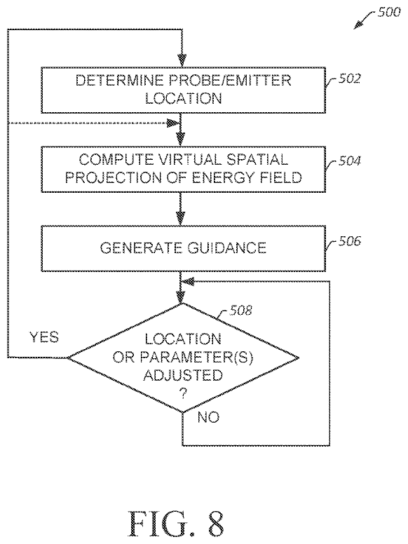

FIGS. 3A and 3B are schematic illustrations showing an example of using of an ablation catheter 200. FIGS. 3A and 3B illustrate an example graphical visualization in which a graphical rendering of a virtual spatial projection of the field superimposed on a graphical representation of the ablation catheter 200 and patient anatomy (e.g., based on navigation and/or geometry data). The visualizations shown in FIGS. 3A and 3B are useful examples that can be generated by the systems and methods described herein (e.g., FIGS. 1 and 8), and reference can be made to such descriptions for additional information.

In the example of FIGS. 3A and 3B, the ablation catheter 200 is configured to perform PFA ablation), such as for creating lesions in tissue 202, such as at the ostium 204 adjacent the left superior pulmonary vein. In an example, the catheter 200 can be the Pulmonary Vein Ablation Catheter GOLD (PVAC™ GOLD) ablation catheter available from Medtronic plc. In the example of FIGS. 3A and 3B, the catheter 200 includes ring-shaped electrode structure 206 having a plurality of electrodes 208. In other examples, the catheter 200 can have other shapes and configurations. In some examples, the electrodes 208 can be configured for delivering energy. The field energy can be delivered by one or more electrodes that make contact with tissue. In other ablation modalities (e.g., PFA), energy can be delivered to tissue in a non-contact manner. In addition, or as an alternative example, the catheter 200 can include one or more of the electrodes 208 and/or one or more other sensors (not shown-see, e.g., other sensors 22 of FIG. 1) configured to measure one or more physiological or device conditions, including one or more electrophysiological signals (e.g., unipolar or bipolar electrograms), environmental conditions (e.g., temperature and/or pressure) and/or contact between the tissue and the electrodes 208 or another part of the catheter. Such measured physiological or device conditions can be used by the systems and methods described herein for mapping and/or validating results of ablation.

As described herein, a navigation system can provide location data representative of coordinates for the catheter 200. The systems and methods here determine a location of the probe relative to patient anatomy based on location data and geometry data. A virtual spatial projection of an energy field the electrode structure can be generated for the electrode 206 based on the location of the electrode relative to the patient anatomy, emitter data and operating parameter(s) for the PFA ablation. In this example, the emitter data can describe energy field properties for the multi-electrode ring structure, which data can be determined a priori, such as being provided a manufacturer, derived from empirical studies and/or field modeling software. Output guidance can be generated and rendered on a display based on the virtual spatial projection, such as to provide a graphical map adapted to visualize a graphical representation of a 3D virtual field projection 210 overlaid on a graphical representation of anatomy, such as shown in FIG. 3A surrounding the electrode. As described herein, a user can change the position of the catheter 200 and/or tune operating parameters of the catheter 200 (e.g., pulse interval, pulse width, number of pulses, and/or field strength) which will trigger updates to the graphical representation of a 3D virtual field projection 210. In some examples, a user can change the viewing angle in response to a user input, such as to visualize virtual penetration of the field into the tissue. Additionally, contact sensing or an imaging modality (e.g., fluoroscopy, intracardiac echo, etc.) can be used to confirm contact between the electrodes and target tissue.

As shown in FIG. 3B, once the user is satisfied with the location and distribution of the 3D field projection relative to the target tissue (and any non-target tissue), the user can trigger the control system (e.g., using a button, switch or other user interface component, such as a graphical user interface element, such as part of user interface 50) to cause energy delivery using the operating parameters set by the user to ablate the tissue accordingly, shown at 212 as the lighter colored ring beneath the electrode 206. For example, the guidance generator can provide a warning and/or specify a distance between a field projection or probe and any defined non-target that should be avoided. The warning can be generated if the field projection or probe is within a threshold distance from the non-target. The threshold distance can be a default value or be user programmable in response to user input.

Also shown in FIG. 3B are additional graphical display windows 214 that can be rendered along with the visualization of on one or more displays. For example, the graphical display windows 214 can include a display of measured electrophysiological signals, such as measured by electrodes 208 or other sensors before, during or following tissue ablation. The graphical display windows 214 can include a graphical user interface for display controls, such as to control animation and other display features. In some examples, the graphical user interface can also be configured to adjust one or more ablation parameters and/or to deliver energy for ablation (e.g., responsive to activating a GUI button).

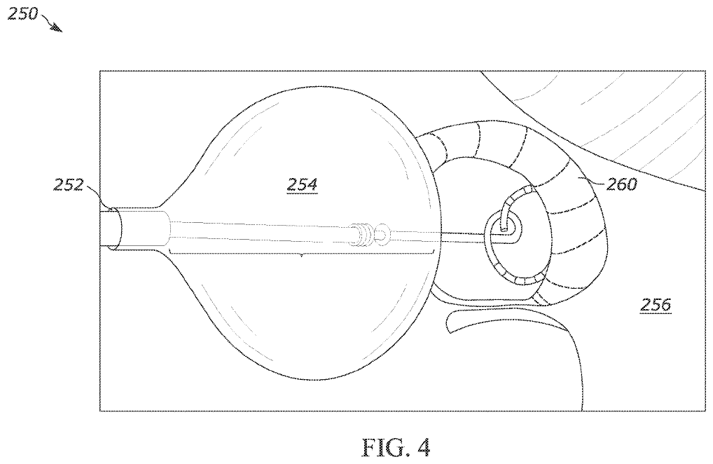

FIG. 4 is a schematic illustration showing an example of a graphical representation 250 of output guidance that can be provided during use of a cryoablation catheter 252, such as can be provided (e.g., as output data) to display 58 by output generator 54. For example, the catheter 252 includes a cryoballoon 254 configured to apply thermal energy (e.g., cooling) for creating lesions in tissue 256, such as at the ostium adjacent the left superior pulmonary vein. As an example, the catheter 252 can be implemented using a catheter selected from the Arctic Front™ family of cardiac cryoablation catheters commercially available from Medtronic plc. As described herein, systems and methods can generate output guidance on a display console, such as to visualize a graphical representation of a 3D virtual field projection 260 overlaid on a graphical representation of anatomy based on the location of the cryoballoon 254 (e.g., determined by location calculator from location data) and selected operating parameters (e.g., size, temperature, etc.). For example, the virtual spatial projection of an energy field can be determined for the cryoballoon 254 based on the location of the cryoballoon relative to the patient anatomy, cryo-emitter data and operating parameter(s) for the cryoablation. In this example, the cryo-emitter data can describe cryo energy field properties for the cryoballoon structure, which data can be determined a priori, such as being provided a manufacturer, derived from empirical studies and/or field thermal modeling software. Additionally, contact sensing or an imaging modality (e.g., fluoroscopy, intracardiac echo, etc.) can be used to confirm contact between the electrodes and target tissue.

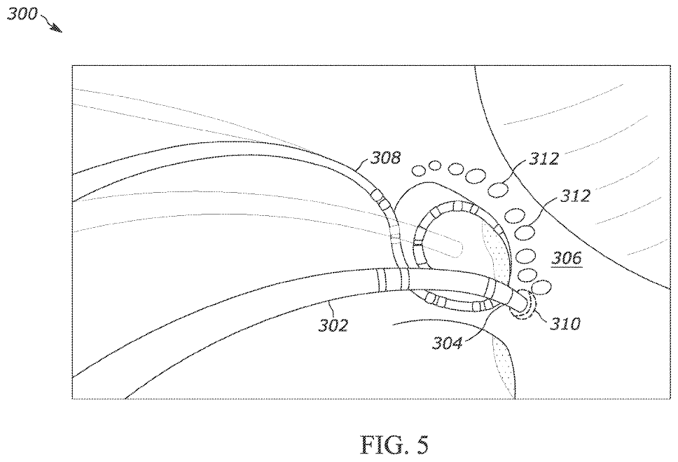

FIG. 5 is a schematic illustration showing an example of a graphical representation 300 that can be provided (e.g., by output generator 54 to display 58) during use of an ablation catheter represented at 302. For example, the catheter 302 includes a tip element 304 configured to apply energy (e.g., RF energy or cooling energy) for creating focal lesions in tissue 306, such as at the ostium adjacent the left superior pulmonary vein. As an example, the catheter 302 can be implemented using one of the RF Marinr™ MC catheters commercially available from Medtronic plc. As another example, the catheter 302 can be implemented using one of the Freezor™ family of cardiac cryoablation catheters commercially available from Medtronic plc.

In the example of FIG. 5, a mapping catheter 308 is used in conjunction with the catheter 302, such as for measuring electrophysiological signals. As described herein, systems and methods can generate output guidance on a display console, such as to visualize a graphical representation of a 3D virtual field projection 310 overlaid on a graphical representation of anatomy based on the location of the catheter tip 304 and selected operating parameters (e.g., tip diameter, tip length, temperature, etc.). For example, the virtual spatial projection of an energy field can be determined for the tip 304 based on the location of the catheter tip relative to the patient anatomy, emitter data and operating parameter(s) for the ablation. In the example of RF ablation, the emitter data can describe RF field and thermal properties for the ablation electrode(s) over a range of respective operating parameters. In the example of cryoablation, the emitter data can describe cryo energy field properties for the tip structure. The emitter data can be determined a priori, such as being provided a manufacturer, derived from empirical studies and/or field thermal modeling software. Also shown in FIG. 5 are previously ablated target sites 312.

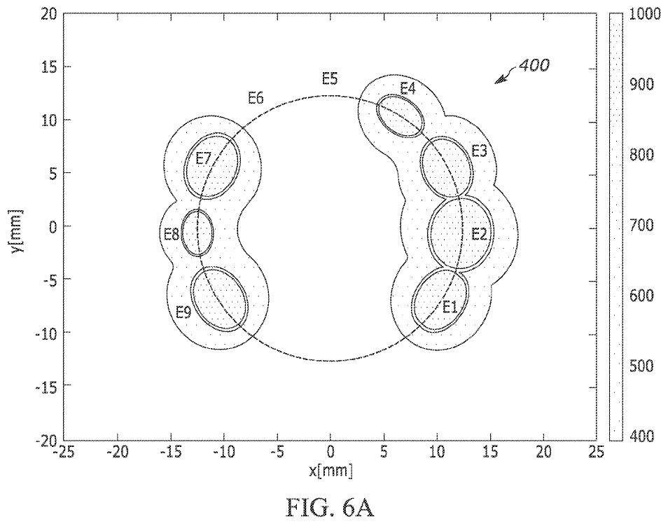

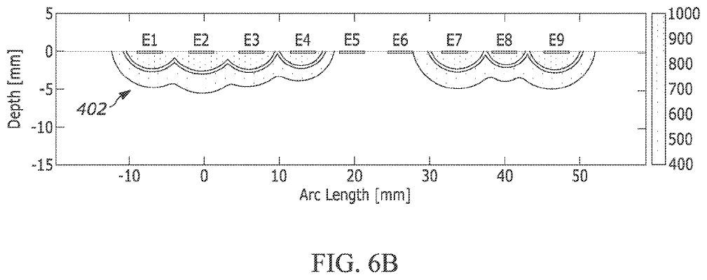

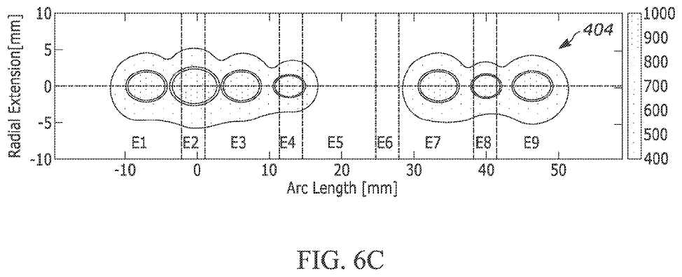

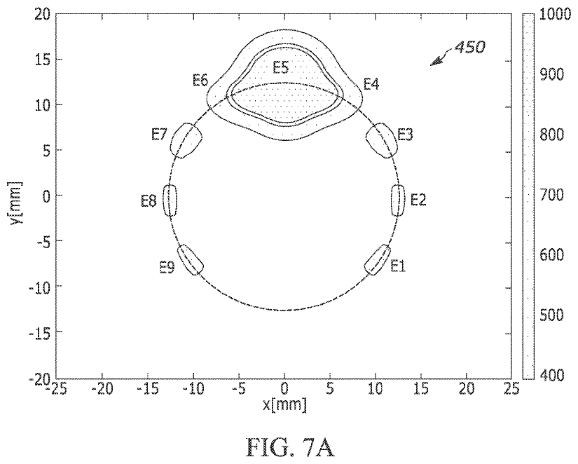

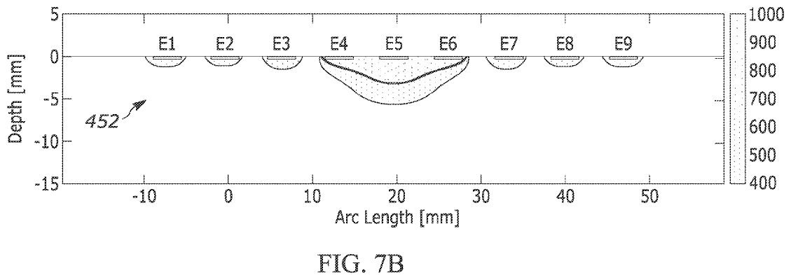

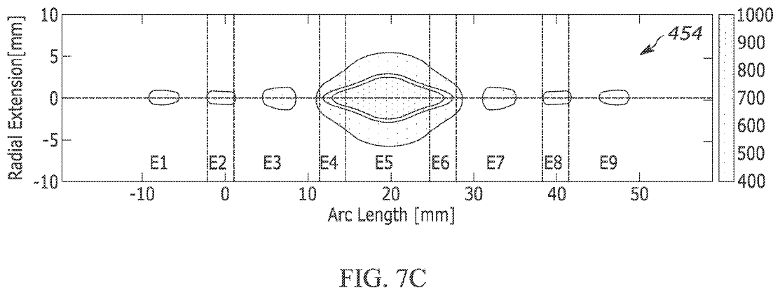

FIGS. 6-7 are graphs showing examples of energy fields that can be provided for different configurations of ablation electrodes and operating parameters. As shown in FIGS. 6-7, the three-dimensional shape (e.g., volume) of the field projection varies depending on which electrodes are energized and the operating parameters. The information provided by such energy fields can be used to provide emitter data 46, which is used to determine virtual field projections described herein. As described herein, any changes in currents, voltages, electrode selections, pulse wave parameters (e.g., pulse width, number of pulses, number of pulse trains), which can be modulated in response to user input selections, can affect virtual field projections. The virtual projection for a given set of ablation parameters can be projected as a graphical representation of a range of voltage isochrones (as V/cm) for each active electrode. Therefore, a threshold can be set to determine if a given field projection would result in unwanted tissue injury (e.g., injury of non-target tissue), and one or more respective parameters can be modulated and the projection dynamically adjusted accordingly to achieve ablation of a desired target region.

For example, FIGS. 6A, 6B and 6C show different views of an electric field projection, shown at 400, 402 and 404, for a ring electrode having nine electrodes shown as electrodes E1-E9. In the example of FIGS. 6A, 6B and 6C, the electrodes E5 and E6 are inactive and a relatively uniform energy level is provided to electrodes E1-E4 and E7-E9. For such electrode configuration and operating parameters, view 400 shows a top view of the electric field projections. View 402 shows a depth profile of the field projections, and view 404 shows a side view showing a width profile of the field projections.

FIGS. 7A, 7B and 7C show different views of an electric field projection, shown at 450, 452 and 454, for a ring electrode having nine electrodes shown as electrodes E1-E9. In the example of FIGS. 7A, 7B and 7C, the electrodes E1-E4 and E6-E9 are configured to deliver energy to electrode E5, which is operating in focal mode on the loop catheter. Given such electrode configuration and operating parameters, view 450 shows a top view of the electric field projections, view 402 shows a depth profile of the field projections, and view 404 shows a side view showing a width profile of the field projections.

FIG. 8 depicts examples of a method 500 that can be implemented by the system 10 to perform respective functions herein. While for purposes of simplicity of explanation, the example method of FIG. 8 is shown and described as executing serially, the example method 500 is not limited by the illustrated order, as some actions could in other examples occur in different orders, multiple times and/or concurrently from that shown and described herein. Additionally, each of the method 500 can be implemented as machine-readable instructions executed by a processor, such as by the mapping system 20. Accordingly, the description of FIG. 8 and also refers to FIG. 1.

Referring to FIG. 8, the method 500 begins at 502 in which a location of a probe/emitter 12 is determined (e.g., by location calculator 40) relative to patient anatomy 14. As described herein, the probe includes an emitter adapted to deliver energy. At 504, the method includes computing (e.g., by field projection calculator 44) a virtual spatial projection of an energy field for the emitter based on the location of the probe and at least one operating parameter for the emitter. The field projection can be computed based on emitter data, such as describing a model, a look-up table or other function or data structure programmed to generate a field projection.

At 506, the method includes generating (e.g., by guidance generator 52) guidance for performing an intervention with the probe based on the virtual spatial projection. As described herein, the guidance can be rendered (e.g., by output generator) to include a rendering of a graphical map 60 visualizing a graphical representation of the virtual field projection superimposed relative to a graphical representation of the patient's anatomy. Also, the visualization can include one or more identified tissue regions, such as including one or more target regions and/or non-target regions.

From 506, the method proceeds to 508 to determine if the location of the probe and/or one or more operating parameters have been adjusted. Responsive to determining the location of the probe and/or one or more operating parameters have been adjusted, the method returns to 502 to update (e.g., re-compute) the respective computations at 502-506 for determining a field projection model and updated user-perceptible guidance. In an example, where the location of the emitter remains unchanged but one or more parameters are changed the method can return to 504 for computing an updated field projection and guidance. If neither the location nor any parameters are adjusted, the method 500 can loop at 508. The method 500 can continue until terminated by the user, at any time, such as after the guidance derived from the virtual projection is within expected/desired parameters and one or more interventions are performed. The method 500 can also include other features shown and described herein.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

As used herein, the recitation “based on” means “based at least in part on.” Therefore, if X is based on Y, X may be a function of Y and any number of other factors.

Various examples have been described. These and other examples are within the scope of the following claims.

1. One or more non-transitory machine-readable media having instructions, which, when executed by a processor, cause the processor to perform a method comprising:
determining a location of a probe relative to patient anatomy, the probe comprising an emitter adapted to deliver energy;
computing a virtual spatial projection of an energy field for the emitter based on the location of the probe and at least one operating parameter for the emitter; and
generating guidance for performing an intervention with the probe based on the virtual spatial projection.

2. The media according to claim 1, wherein the method further comprises setting the at least one operating parameter in response to a user input, and
wherein the computing and the generating are repeated based on the setting of the at least one operating parameter.

3. The media according to claim 1, wherein:
the virtual spatial projection comprises a three-dimensional volume representing an electric field that varies based on the at least one operating parameter, tissue properties for surrounding tissue, and a distance from the emitter,
the method further comprises:
defining at least one non-target region within the patient anatomy, and
generating the guidance further comprises generating a graphical visualization that includes the virtual spatial projection, a graphical representation of the at least one non-target region and a graphical representation of a target region within the patient anatomy.

4. The media according to claim 3, wherein the method further comprises:
adjusting the at least one operating parameter of the emitter in response to determining that the at least one non-target region resides within the three-dimensional volume of the virtual spatial projection; and
updating the three-dimensional volume for the virtual spatial projection based on the location of the probe and the adjusted at least one operating parameter for the emitter; and
updating the guidance based on the updated three-dimensional volume for the virtual spatial projection.

5. The media according to claim 1, wherein the at least one operating parameter of the emitter is adjusted automatically so the target region resides within the three-dimensional volume of the virtual spatial projection and the at least one non-target region resides outside of the three-dimensional volume of the virtual spatial projection.

6. The media according to claim 3, wherein the at least one non-target region is defined in response to a user input.

7. The media according to claim 3, wherein the guidance further comprises generating an output to indicate whether the at least one non-target region resides within the three-dimensional volume of the virtual spatial projection.

8. The media according to claim 1, wherein:
the emitter comprises a plurality of electrodes in a distributed spatial arrangement,
each of the plurality of electrodes is configured to deliver energy based on respective operating parameters, and
the virtual spatial projection comprises a three-dimensional volume representative of an aggregate electric field emanating from the each of the plurality of electrodes based on the respective operating parameters.

9. The media according to claim 1, wherein the virtual spatial projection of the energy field is computed based on a type the energy the emitter is configured to deliver, and the emitter is configured to deliver the energy using one of radiofrequency ablation, cryoablation, laser ablation or pulsed field ablation.

10. The media according to claim 1, wherein the method further comprises controlling the energy delivered by the emitter to a target region based on the guidance and the at least one operating parameter.

11. The media according to claim 10, wherein:
the virtual spatial projection comprises a three-dimensional volume, the method further comprises defining at least one target region within the patient anatomy, and
generating the guidance further comprises generating a graphical visualization that includes the virtual spatial projection and a graphical representation of the at least one target region within the patient anatomy.

12. The media according to claim 10, wherein the method further comprises:
storing electrophysiological data representative of electrophysiological signal measurements over a first time interval before delivery of the energy and a second time interval after delivery of the energy; and
confirming a desired therapeutic or subtherapeutic effect based on a comparison of the electrophysiological data measured before delivery and after delivery of the energy.

13. The media according to claim 1, wherein multiple virtual spatial projections are computed for different values of the at least one operating parameter for the emitter and/or locations of the probe, and the method further comprises:
selecting a given virtual spatial projection from among the multiple virtual spatial projections in response to a user input;
setting the at least one operating parameter to a corresponding value based on the selected given virtual spatial projection; and
controlling energy delivery from the emitter based on the setting.

14. A system, comprising:
an elongated probe comprising an emitter adjacent a distal end thereof, the emitter configured to deliver energy based on at least one operating parameter thereof;
non-transitory memory configured to store data and machine-readable instructions;
one or more processors adapted to access the memory and execute the instructions programmed to cause the processor to at least:
determine a location of the probe relative to patient anatomy based on location data and geometry data, the location data representing spatial coordinates of the probe, and the geometry data spatially representing at least a target region of the patient anatomy;
determine a virtual spatial projection of an energy field for the emitter relative to the patient anatomy based on the location of the probe, emitter data and the at least one operating parameter for the emitter, the emitter data describing energy field properties for the emitter; and
generate guidance based on the virtual spatial projection.

15. The system of claim 14, further comprising setting the at least one operating parameter in response to a user input,
wherein the virtual spatial projection and the guidance are updated responsive to the user input.

16. The system according to claim 14, wherein:
the instructions further programmed to define at least one non-target region within the patient anatomy, and
the guidance further comprises a graphical visualization that includes the virtual spatial projection, a graphical representation of the at least one non-target region and a graphical representation of a target region within the patient anatomy.

17. The system according to claim 16, wherein the instructions are further programmed to:
adjust the at least one operating parameter of the emitter in response to determining that the at least one non-target region resides within a volume of the virtual spatial projection;
update the virtual spatial projection based on the location of the probe and the adjusted at least one operating parameter for the emitter; and
update the guidance based on the updated virtual spatial projection.

18. The system according to claim 17, wherein the instructions are further programmed to automatically adjust the at least one operating parameter of the emitter so the target region resides within the volume of the virtual spatial projection and the at least one non-target region resides outside of the volume of the virtual spatial projection.

19. The system according to claim 14, wherein the emitter comprises a plurality of electrodes in a distributed spatial arrangement, each of the plurality of electrodes configured to deliver energy based on respective operating parameters, and
wherein the virtual spatial projection comprises a three-dimensional volume representative of an aggregate electric field emanating from the each of the plurality of electrodes based on the respective operating parameters.

20. The system according to claim 14, wherein the instructions are further programmed to control the energy delivered by the emitter to a target region based on the guidance and the at least one operating parameter.
